(19) United States
(12) Patent Application Publication
(10) Pub. No.: US 2026/0042729 A1
(43) Pub. Date: Feb. 12, 2026

(54) FUNCTIONALIZED DIACETYLENE MONOMERS, THEIR POLYMERIZATION AND USES THEREOF

(71) Applicant: B.G. NEGEV TECHNOLOGIES AND APPLICATIONS LTD., AT BEN-GURION UNIVERSITY, Beer Sheva (IL)

(72) Inventors: Raz JELINEK, Modi’in-Maccabim-Re’ut (IL); Rajendran MANIKANDAN, Beer Sheva (IL)

(21) Appl. No.: 19/100,239

(22) PCT Filed: Aug. 3, 2023
(86) PCT No.: PCT/IL2023/050806

§ 371 Date: Jan. 31, 2025

Related U.S. Application Data
(60) Provisional application No. 63/394,646, filed on Aug. 3, 2022.

(51) Int. Cl. C07C 225/22; C07C 221/00; C08F 138/02; C09K 11/06; G01N 21/64; G01N 21/78; G01N 33/02

(52) U.S. Cl. CPC C07C 225/22; C07C 221/00; C08F 138/02; C09K 11/06; G01N 21/6428; G01N 21/78; G01N 33/02; C09K 2211/1425

(57) ABSTRACT

An aminochalcone-diacetylene compound of Formula 1A:

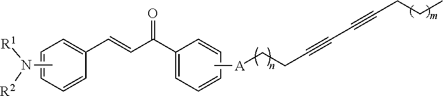

wherein R¹ and R² are independently selected from H and C₁-C₃ alkyl, A denotes a linkage connecting the chalcone and diacetylene units, m and n are independently integers in the range of 2 to 18, and the corresponding protonated form/ammonium salt of Formula 1B:

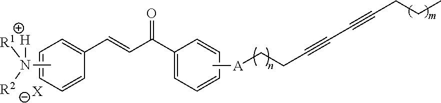

wherein X is a counter anion. The disclosure further discloses the corresponding chromatic polydiacetylenes and polydiacetylenes-based sensors for detecting ammonia.

### CROSS-REFERENCE TO RELATED APPLICATIONS

This application is a National Stage application of International Patent Application No. PCT/IL2023/050806, filed on Aug. 3, 2023, which claims priority to U.S. Provisional Patent Application No. 63/394,646, filed on Aug. 3, 2022, each of which is hereby incorporated by reference in its entirety.

### TECHNICAL FIELD

The present disclosure relates to functionalized diacetylene monomers, their polymerization and uses thereof.

### BACKGROUND

Polydiacetylenes (PDAs) are π-conjugated organic polymers synthesized by crosslinking diacetylene (1,3-butadiyne)-based monomers. Alignment of the diacetylene units is an essential precondition for the occurrence of the photoinduced topochemical polymerization of PDA systems, which exhibit remarkable colorimetric and fluorescence properties. As polymerized PDA generally appears blue due to the conjugated PDA network, it transforms to red when subjected to external stimuli such as pH, temperature, mechanical strain, and molecular interactions. In parallel, PDA systems display interesting fluorescence properties, as the blue PDA phase is non-fluorescent while the red phase exhibits intense fluorescence emission.

The optical transitions of PDA have been employed in diverse optical and sensing applications. For example, PDA-based colorimetric and fluorescent sensors for bacteria and food spoilage have been reported [L. H. Nguyen, S. Naficy, R. McConchie, F. Dehghani and R. Chandrawati, J. Mater. Chem. C, 2019, 7, 1919-1926 and S. Park, G. S. Lee, C. Cui and D. J. Ahn, Macromol. Res., 2016, 24, 380-384]. Other examples in the field of bacteria sensing include U.S. Pat. No. 6,361,962 (a multi-type toxin indicator comprising polydiacetylenes for detecting the presence of bacteria in foods) and US 2002/0034475, which discloses the incorporation of polydiacetylenes into various food products; the PDAs undergo a chromatic colour transition in response to various triggering mechanisms such as temperature change, pH change, mechanical stress and the presence of bacteria in the food.

Diacetylene monomers can be readily functionalized, and substitutions of diacetylene headgroups have been employed for modulating the structural and optical properties of PDA assemblies. The functionalization of PDA with optically active and fluorescent moieties is particularly interesting as such systems furnish diverse optical properties and applications. For example, attachment of dansyl and BODIPY fluorophores to the PDA system resulted in the amplification of the fluorescence quantum yield of the red phase PDA via FRET transfer after thermal treatment, reactions with target analytes [X. Li, S. Matthews and P. Kohli, J. Phys. Chem. B, 2008, 112, 13263-13272 and G. Ma, A. M. Müller, C. J. Bardeen and Q. Cheng, Adv. Mater., 2006, 18, 55-60]. Similar FRET-based sensitivity enhancement was reported with other fluorescent substituents [M. A. Reppy and B. A. Pindzola, Chem. Commun., 2007, 4317-4338]. Yet in another application, linking the perylene diimide (PDI) chromophore to PDA magnified photocurrent generation and the PDI-PDA conjugate was utilized in flexible supercapacitors [J. Seo, C. Kantha, J. F. Joung, S. Park, R. Jelinek and J. M. Kim, Small, 2019, 15, 1-8 and A. De Adhikari, A. Morag, J. Seo, J. M. Kim and R. Jelinek, ChemSusChem, 2020, 13, 3230-3236]. Recently developed anthraquinone functionalized PDA assemblies manifested interesting vapor sensing and mechano-chromic properties [R. Bisht, V. Dhyani and R. Jelinek, Adv. Opt. Mater., 2021, 9, 1-10].

### SUMMARY

Chalcones are π-conjugated molecules of the structure:

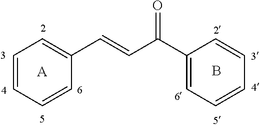

Experimental work reported below shows a reaction between a diacetylene monomer and (N-alkylated) aminochalcones, to give the corresponding amine-substituted chalcone-diacetylene monomer. A thin film of the monomer was produced by solution casting and was then exposed to the action of different gaseous acids, to bring about the protonation of the amine and formation of the corresponding ammonium salt. Depending on the counter anion delivered by the acid, the ammonium salt of the chalcone-diacetylene monomer can undergo UV polymerization to give a colored polydiacetylene. The so-formed colored polydiacetylene exhibits unique color and fluorescence properties. The chalcone-polydiacetylene responds selectively to the presence of ammonia vapors, showing rapid, pronounced colorimetric and fluorescence changes, even at low temperatures. Owing to its responsiveness to ammonia vapors—a prominent volatile metabolite secreted by bacteria—the chalcone-polydiacetylene system can be used for visible bacterial sensing and food spoilage monitoring.

Thus, a first aspect of the invention relates to amino chalcone-diacetylene of Formula 1A:

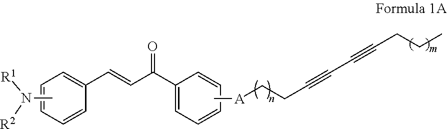

wherein R¹ and R² are independently selected from H and C₁-C₃ alkyl, A denotes a linkage (e.g., an ester bond or an amide bond) connecting the chalcone and diacetylene units, and m and n are independently integers in the range of 2 to 18, (e.g., 5 to 10), and the corresponding protonated form/ammonium salt of Formula 1B:

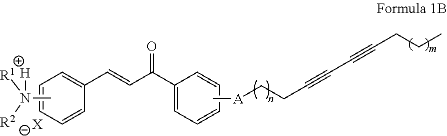

wherein X is a counter anion. The compound of Formula 1B is photopolymerizable into a colored PDA.

The amine-substituted chalcone-diacetylene of Formula 1A is prepared by the reaction between a diacetylene compound of Formula 2 and aminochalcone of Formula 3:

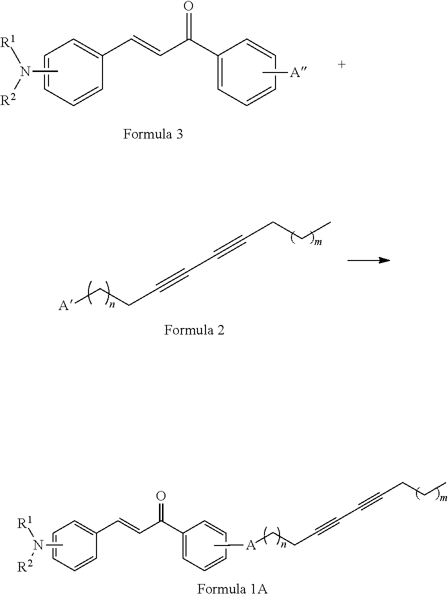

wherein n, m, R¹ and R² are as defined above, A′ and A″ denote functional groups which can participate in a linkage formation reaction, to create a linkage denoted by the letter A in compounds of Formula 1A and 1B. The linkage A is preferably an ester bond or an amide bond; most preferred is the ester bond:

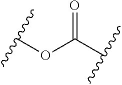 [SMILES: *OC(*)=O]

The —NR¹R² group and the A linkage are preferably attached at the para positions of the respective phenyl rings of the chalcone system. N,N-dialkylated (e.g., dimethylated) derivatives are especially useful; the most preferred monomer of Formula IA has the structure depicted below:

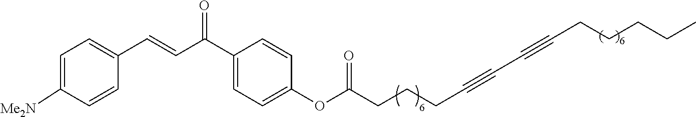

This monomer is labeled herein CHA-DA.

Starting with the diacetylene monomers of Formula 2:

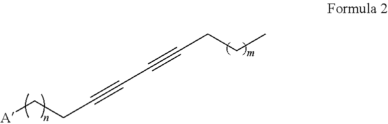

they are commercially available in the form of their carboxylic acid derivatives, i.e., A′=—COOH, e.g., 10,12-tricosadiynoic acid (TRCDA), 10,12-pentacosadiynoic acid, 10,12-octadecadiynoic acid, 5,7-docosadiynoic acid, 5,7-pentacosadiynoic acid and 5,7-tetracosadiynoic acid. The carboxylic acids are readily transformed into the corresponding acyl halide (—COCl). The acyl halides are usually more reactive reagents than the parent carboxylic acids and are more favorable for use in the invention. Acyl chloride of carboxylic acid is normally prepared by dissolving the carboxylic acid in an organic solvent, such as dichloromethane, using reagents such as oxalyl chloride or thionyl chloride. The reaction is advanced by addition of a small, catalytically effective amount of dimethylformamide. The acyl chloride derivative of Formula 2 can be recovered after the removal of the solvents and excess reagent, for use in the preparation of the compound of Formula 1A. Another option is to convert carboxylic acid diacetylene (A′=—COOH) to an amide derivative bearing a functional side group, such as amine (as described, e.g., in http://amsdottorato.unibo.it/7925/1/Beglaryan_Stella_tesi.pdf).

Turning now to the aminochalcones, their N-monoalkylated and N,N-dialkylated derivatives are represented by Formula 3:

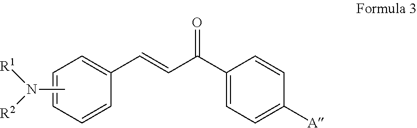

Suitable starting materials of Formula 3 are described, for example, in a review article [R. Irfan, S. Mousavi, M. Alazmi and R. S. Z. Saleem, Molecules, 2020, 25 (22), 5381].

A convenient method of synthesizing the compounds of Formula 3 is through the base-catalyzed reaction of an aldehyde and ketone, i.e., the Claisen-Schmidt procedure, e.g., N,N-dialkylated-aminobenzaldehyde is reacted with acetophenone bearing the group A″, using sodium hydroxide as the base, in ethanol, at room temperature [B. Korkmaz, E. A. Özeroln, Y. Gürsel, B. F. Senkal, M. Okutan, J. Mol. Liq., 2018, 266, 132-138]:

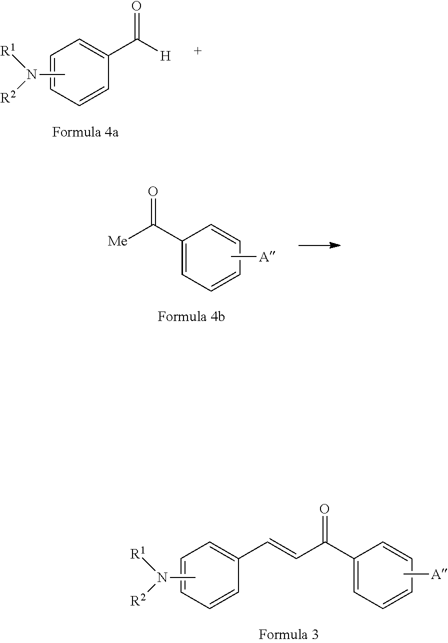

The preferred compounds of Formula 3 for use in the invention are 4-aminochalcone, N-monoalkylated (e.g., methylated) 4-aminochalcone and N,N-dialkylated (e.g., dimethylated) 4-aminochalcone, with the A″ group being selected from hydroxyl (—OH) and carboxylic acid derivatives (e.g., acyl halide —C(O)Cl), attached at position 4′ of the chalcone, leading to the creation of ester or amide bond, respectively, between the chalcone of Formula 3 and diacetylene compound of Formula 2.

The preparation of the most preferred compound of Formula 3:

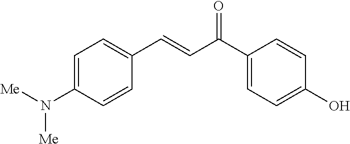 [SMILES: CN(C)c1ccc(/C=C/C(=O)c2ccc(O)cc2)cc1]

Chemical name: (E)-3-(4-(dimethylamino)phenyl)-1-(4-hydroxyphenyl) prop-2-en-1-one (labeled herein CHA) is shown in the experimental section below.

For example, an ester formation reaction of the monomer of Formula 1A from the acyl chloride of Formula 2 and the alcohol of Formula 3:

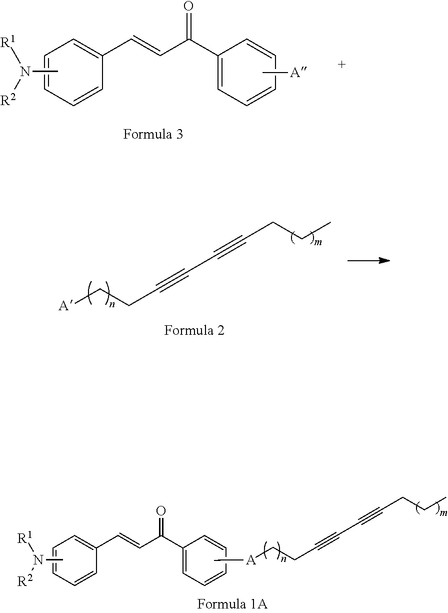

(i.e., A′ is —C(O)Cl, A″ is —OH and A is —C(O)—O—), takes place in an organic solvent, e.g., an halogenated hydrocarbon such as dichloromethane, at room temperature, and is catalyzed by an organic amine, e.g., trialkyl amine. For example, a solution of the acyl chloride of Formula 2 is gradually added to a reaction vessel that was previously charged with a solution of alcohol of Formula 2 and triethyl amine. After the addition of the acyl chloride of Formula 2 is completed, the reaction is maintained under stirring for an additional period of time, at room temperature to bring the reaction to completion. The chalcone-diacetylene monomer of Formula 1A is then isolated from the reaction mixture, e.g., by solvent evaporation, in the form of a solid exhibiting pale yellow color, characteristic of the chalcone system.

Thus, another aspect of the invention is a process comprising a reaction between a diacetylene monomer of Formula 2 and an aminochalcone of Formula 3, as depicted below:

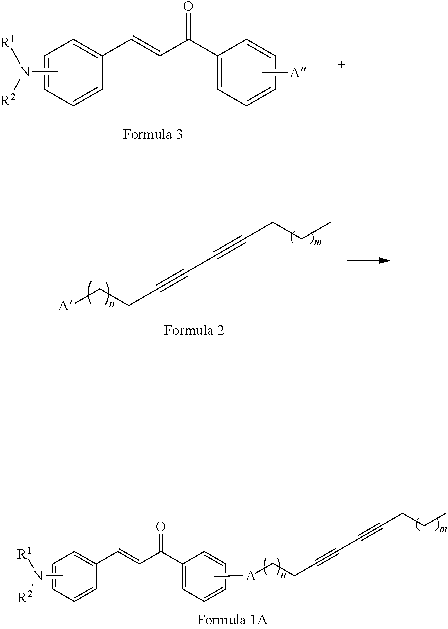

and specifically, an amine-catalyzed ester formation reaction wherein A″ is OH, A′ is —C(O)Cl and A is —C(O)—O—.

Diacetylene monomer assembly normally undergoes UV polymerization to form colored PDA, but it turned out not to be the case for the monomer of Formula 1A. Assembling the chalcone-diacetylene monomer of Formula 1A into a thin film by solvent casting, followed by irradiation of the film with UV light (at 254 nm), failed to produce a colored PDA system. However, exposure a chalcone-diacetylene monomer assembly of Formula 1A to the action of suitable acid vapors resulted in the formation of the corresponding protonated form/ammonium salt of the chalcone-diacetylene monomer (the terms are used herein interchangeably), represented by Formula 1B:

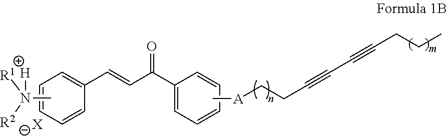

which turned out to be UV-polymerizable into purple colored PDA at room temperature. Specifically, experimental work reported below shows that by the action of vapors of hydrohalic acids (HX, wherein X is halide, especially gaseous HCl) on a solvent-casted, yellow-colored chalcone-diacetylene film, transformation into a colorless film occurs. Spectroscopic analysis of the colorless film indicates that the amine group attached to the aromatic ring has undergone protonation. FTIR spectrum shows a new strong peak at around 3350 cm⁻¹, assigned to the protonated N—H group. UV-vis spectrum of the colorless film shows that the peak at around 420 nm, characteristic of the chalcone system, has completely disappeared, attesting to the blocking of charge transfer within the chalcone system due to the protonation of the dimethylamine unit.

Thus, another aspect of the invention is a process comprising assembling chalcone-diacetylene monomers of Formula 1A (e.g., into a thin film or water-suspended vesicles) and treating the monomer assembly (e.g., the thin film) with gaseous hydrohalic acid, e.g., HCl vapors, to obtain the corresponding protonated form/ammonium salt:

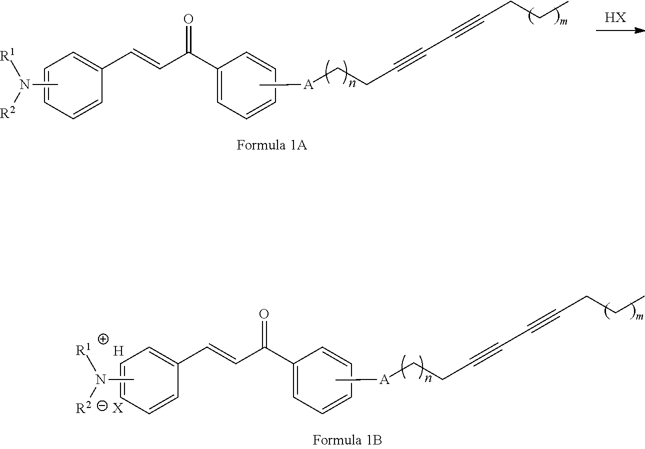

Creation of monomer assembly by deposition of chalcone-diacetylene films (i.e., films consisting of the compound of Formula 1A) onto a suitable substrate (made of glass, plastic, or filter paper, to name a few examples) can be achieved by any acceptable technique, such as solvent casting and spin coating.

For example, dichloromethane, owing to its ability to solubilize the chalcone-diacetylene of Formula 1A and its high volatility, is well suited for use in creation of thin films by solvent casting. A solution of 5-20 (mg/mL) of the chalcone-diacetylene of Formula 1A is prepared and casted on the substrate (e.g., by drop-casting on a laboratory scale or doctor blade casting on a large scale), following by evaporation of the dichloromethane solvent, to create 1-100 μm thick films. Other organic solvents that may be used are chloroform, ethyl acetate and acetonitrile.

As pointed out above, the as-deposited film of the compound of formula 1A has yellow color (due to absorption in the violet region of the chalcone units and the appearance of corresponding complementary yellow color). The yellow color disappears when the film is exposed to gaseous acid, e.g., when it is held in an environment created by the flow of acid vapors over the surface of the film. For example, on a laboratory scale, this can be achieved fairly easily by pouring a concentrated solution of hydrochloric acid into a vessel and placing the film a few centimeters above the vessel for one or two minutes (each of the opposite faces of the film is treated in this manner). It is also possible to spread a very small volume of the aqueous HCl directly onto the film, whereby the film is exposed to HCl gas released from the solution. That is, vapors of an acid with a relatively small counter anion, namely, HX when X is halide, specifically chloride, whereby a colorless film is formed. The color change (more precisely, disappearance of yellow color) in response to the presence of HCl vapors suggests that a film made of the compound of formula 1A is useful on its own right as a sensor for detecting HCl leakage, e.g., in industrial plants. A solid-state sensor of HCl gas, comprising a compound of formula 1A, e.g., in the form of a film deposited on a substrate, constitutes another aspect of the invention.

But the major feature of the acid-treated monomer assembly, consisting of the protonated form/ammonium salt represented by Formula 1B, resides in its polymerizability: under UV irradiation, colored PDA is created, as shown below:

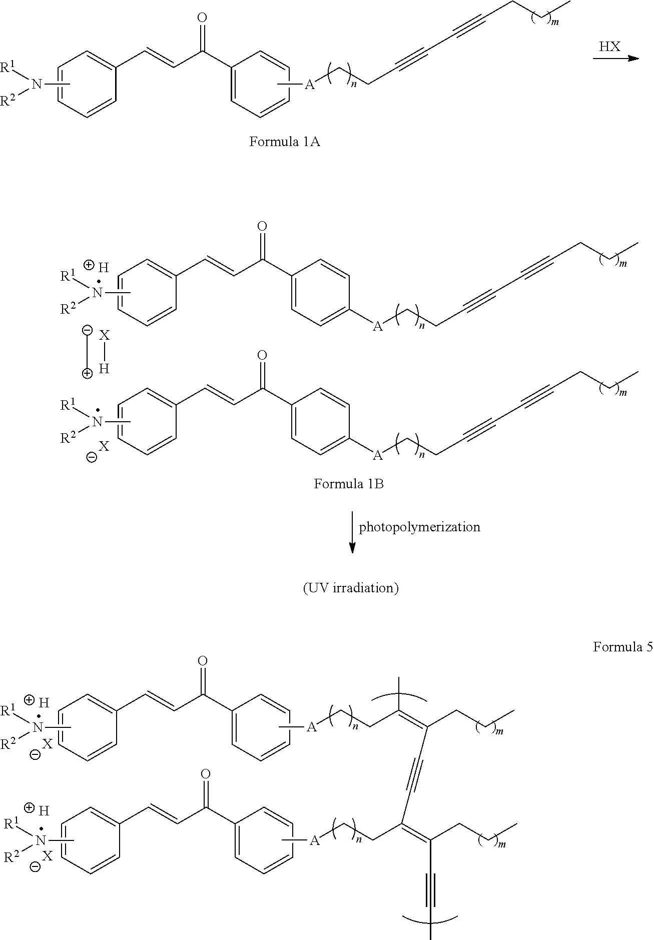

Thus, a polydiacetylene incorporating chalcone pendant groups, with protonated amino groups/ammonium halide groups attached to a benzene ring of the chalcone system, e.g., the chalcone-polydiacetylene of Formula 5, wherein R¹, R², A, n and m are as previously defined, forms another aspect of the invention.

In the preferred chalcone-polydiacetylene of Formula 5, R¹ and R² are both methyl, A is an ester bond —O—C(O)—, n equals 6, m equals 8, and X is chloride. This specific polymer, which is shown in FIG. 1C below, is labeled herein CHA-PDA-HCl.

The chalcone-polydiacetylene of Formula 5 was tested to evaluate its ability to sense various analytes in the gaseous state and was shown to selectively detect vapors of ammonia and related amine compounds, as opposed to vapors of organic solvents devoid of basic nitrogen functionality, such as hexane, toluene, dichloromethane, chloroform, tetrahydrofuran (THF), dimethylformamide (DMF), dimethyl sulfoxide (DMSO) and ethanol. In the presence of ammonia vapors, the chalcone-polydiacetylene of Formula 5 (with its characteristic purple color at room temperature), exhibits purple-orange visible color transition and a blue-yellow/pastel-orange fluorescence transformation (excitation at 365 nm) at 20° C.

It appears that the ammonium group attached to the chalcone-polydiacetylene of Formula 5 undergoes deprotonation in the presence of ammonia molecules, i.e., ammonia molecules capture the acidic proton bound to the —HN⁺R¹R² group with concomitant release of the cognate halide (e.g., Cl⁻) ions, such that the nitrogen atom in (alkylated) amino group —NR¹R² regains its lone electron pair, and its ability to function as an electron donor, reintroducing charge transfer from the —NR¹R² groups to the carbonyl units (electron acceptors). The result is that the chalcone system reacquires its characteristic yellow color. In parallel, ammonia induced the phase transformation within the conjugated PDA network giving rise to a blue-red transition of PDA. Consequently, a blending of the yellow color of the chalcone residue and red PDA (at 20° C.) generates the orange color, accounting for the purple-orange color transition observed when the chalcone-polydiacetylene of Formula 5 interacts with ammonia vapors at room temperature. As to the fluorescence transformation exhibited by the chalcone-polydiacetylene of Formula 5 upon exposure to ammonia molecules, it is noted that the initial fluorescence is blue. The action of ammonia vapors described above produces pastel orange color, due to the combination of pale-yellow fluorescence of the “reborn” chalcone system and the customary red fluorescence of phase-transformed PDA.

The detection of ammonia vapors by the chalcone-polydiacetylene of Formula 5 is achieved over a broad temperature range, with colorimetric and fluorescence changes occurring in the presence of ammonia vapors at low temperatures, e.g., down to −20° C., because of the deprotonation and release of the counter (halide) anion, as explained above. But due to the significantly constrained motion of the conjugated PDA network at low temperatures, a purple-dark green visible color transition is observed, arising from the blending of the yellow color of the “reborn” chalcone unit and the lavender blue PDA. Similarly, the greenish-yellow fluorescence induced by ammonia is ascribed to the mixing of yellow fluorescence of the chalcone units and the blue-purple emitted fluorescence of the phase PDA, as shown in detail in the experimental section below.

Importantly, the fluorescence emission (excitation at 450 nm) of the chalcone-polydiacetylene of Formula 5 is enhanced with increasing concentration of ammonia vapor molecules, showing a linear relationship in the range of 0.1-100 ppm, and a detection limit of around 3 ppb (corresponding to experimentally significant 1% fluorescence enhancement). Fluorescence enhancement is calculated as follows:

$${F}_{e} = \frac{{F}_{a} - {F}_{b}}{{F}_{a}} \times 100 \qquad \begin{matrix}{F}_{e} = Fluorescence enhancement . \\ {F}_{a} = Fluorescence intensity after exposure . \\ {F}_{b} = Fluorescence intensity before exposure .\end{matrix}$$

Accordingly, the invention relates to a colorimetric and/or fluorescent sensor for detection of vapors of ammonia and related amine compounds, the sensor comprises a polydiacetylene incorporating chalcone pendant groups, with protonated amino groups/ammonium halide groups attached to the chalcone group, e.g., the chalcone-polydiacetylene of Formula 5. By related amine compounds is meant organic ammonia derivatives which are fairly strong bases, with pKa in the range from ˜9 to 10.5, but are relatively non-bulky, e.g., alkylated, for example, methylated ammonia derivatives.

The sensor of the invention lends itself to different applications involving generation and release of ammonia vapors and related amines. That is, the invention provides a colorimetric and/or fluorescent sensor for detection of biogenic ammonia and related amines, produced by microorganism such as bacteria, comprising the chalcone-polydiacetylene of Formula 5 as defined above. Biogenic amines (e.g., ammonia vapors) sensed by the polydiacetylene of Formula 5 indicate the presence of microorganism on a tested material or surface.

For example, because ammonia is a volatile metabolite secreted by bacteria, ammonia can serve as an indicator of food spoilage. The sensor of the invention, in the form of a film made of the HCl-treated chalcone-polydiacetylene of Formula 5 was tested for visual bacterial sensing. The film (deposited on a paper that was attached to a cover of a petri dish, placed ˜1 cm above the surface level of E. coli bacterial cells in a Luria-Bertani (LB) medium) showed color changes in response to evolution of ammonia gas released by the proliferating bacteria in the LB medium (e.g., a light-purple to orange chromatic transition). In addition, a fluorescence response curve established showed correlation between the chromatic changes of the film and bacterial proliferation, i.e., with typical exponential growth curve of bacterial populations.

Importantly, volatile ammonia generated by bacteria proliferating in food products, e.g., fish, stored at 25° C. (room temperature conditions) and 4° C. (refrigerated conditions) was also detected by the sensor of the invention, showing the ability of the ammonia sensor to monitor food spoilage over the relevant temperature range.

Accordingly, another aspect of the invention relates to a colorimetric and/or fluorescent sensor for monitoring food spoilage, the sensor comprises a polydiacetylene incorporating chalcone pendant groups, with protonated amino groups/ammonium halide groups attached to the chalcone group, e.g., the chalcone-polydiacetylene of Formula 5.

One useful design of the colorimetric and/or fluorescent sensor of the invention consists of a thin film of the chalcone-polydiacetylene of Formula 5 film deposited on a substrate produced as described above. An alternative design consists of the polydiacetylene of Formula 5 incorporated into a porous inorganic or organic matrix.

The chromatic transition exhibited by the sensor of the invention in response to low concentration of ammonia vapors (indicating the onset/progress of food degradation) are readily observed by the naked eye over a broad temperature range. Therefore, the sensor, in the form of a thin film supported, e.g., on a flexible plastic sheet, may be incorporated into the packaging of a food product, or may form an integral part of the packaging. The sensor need not be in direct contact with the food product, but accessible to ammonia vapors evolving due to development of bacterial contamination. In this way, possible deterioration of a food product due to bacterial contamination, may be detected as a distinct purple-to-orange color change of the packaging (the exact color transition varies with temperature as described below). To enhance readability, the sensor may be spatially arranged within the food packaging such that upon changing color, a distinct symbol or word becomes visible. Thus, if the sensor of the present invention were to be incorporated in the form of the letter ‘X’ in a portion of the packaging having the same color as the original, protonated sensor, bacterial contamination of the food product would be indicated by the presence of an orange letter ‘X’ set in a purple background.

In circumstances where color changes are not readily visible (e.g., products stored at dark, in refrigerators, during shipping), or in industrial/agrochemical facilities equipped with fluorescent excitation and detection means or a standard fluorescence spectrophotometer, the monitoring of food spoilage can be based on the characteristic fluorescent emission associated with the changes in the polydiacetylene of Formula 5, that occur in response to the presence of volatile (e.g., ammonia vapors) arising from spoilage. Detection of this fluorescent emission may be accomplished by illuminating the film with a suitable light source emitting light at about 350-470, e.g., 430-470 nm (excitation). The appearance of characteristic maxima at about 480-550 nm in the fluorescence spectrum obtained following said excitation serves as an indication for the presence of biogenic amines arising from food spoilage.

The invention further relates to a method for detecting the presence of ammonia vapors, comprising placing the polydiacetylene of Formula 5 (e.g., in the form of a thin film deposited on a substrate) in proximity to a source prone to generating ammonia (e.g., a food product prone to spoilage), and either observing the color of said polydiacetylene of Formula 5 or detecting a fluorescent emission thereof, wherein a change in said color (e.g., purple-orange transition) or a characteristic fluorescence emission indicate the evolution of ammonia vapors.

### BRIEF DESCRIPTION OF THE DRAWINGS

FIGS. 1A-1C show the results of the treatment of CHA-DA films with different acids and subsequent polymerization.

FIGS. 2A-2C show spectroscopic characterization of chalcone-functionalized polydiacetylene. More specifically, FIG. 2A: UV-vis absorption spectra of the non-polymerized and polymerized diacetylene species. FIG. 2B: Raman spectra. The broken vertical lines indicate the (C≡C) and (C═C) stretching frequencies, respectively, in HCl-treated chalcone-PDA. FIG. 2C: FTIR spectra showing N—H vibration frequency domain. The broken vertical line indicates the N—H stretching frequency of chalcone-diacetylene-HCl.

FIGS. 3A-3Cshow the thermochromic properties of the HCl-treated chalcone-PDA film that was deposited on filter paper. More specifically, FIG. 3A: photographs of HCl-treated chalcone-PDA film drop-casted on a filter paper at different temperatures. FIG. 3B: UV-vis absorbance spectra. FIG. 3C: Raman spectra of the HCl-treated chalcone-PDA film at different temperatures.

FIG. 4A shows the color (top row) and fluorescence (bottom row) transformations of the HCl-treated chalcone-PDA film upon brief exposure to ammonia gas at 20° C. and at −20° C. FIG. 4B depicts the deprotonation process caused by the ammonia vapors. FIG. 4C shows the fluorescence modulation induced upon exposure of the HCl-treated polymerized chalcone-PDA film to ammonia vapor, and the linear relationship between the fluorescence enhancement and ammonia concentration. FIG. 4D shows fluorescence enhancement following the action of different organic amines on the PDA film.

FIGS. 5A-5C show photos of a visual bacterial sensing test of the HCl-treated chalcone-PDA films, and fluorescence enhancement versus bacteria cell/ml curve.

FIGS. 6A-6B show photos of HCl-treated chalcone-PDA films, sensing ammonia generated by bacterial proliferation in fish samples.

### DETAILED DESCRIPTION

### Materials

4-dimethylaminobenzaldehyde was purchased from Sigma Aldrich (Bangalore, India), and 4-hydroxyacetophenone was purchased from Sigma Aldrich (Shanghai, China). Sodium hydroxide and organic solvents including hexane, dichloromethane, chloroform, acetone, ethyl acetate and ethanol were purchased from Bio-Lab Ltd. (Jerusalem, Israel). All these chemicals were used without further purification. 10,12-tricosadiynoic acid (TRCDA) was purchased from Alfa Aesar (Lancashire, England) and purified prior to use by dissolving in chloroform and passing using a 0.8 μm syringe filter followed by solvent removal by rotary evaporation.

### Methods

Ultraviolet-visible (UV-vis) spectra: The samples for thin-film measurements were prepared by drop-casting 50 μL of 15 mg/mL solution of the desired compound onto glass substrates. UV-vis spectra were recorded on an Evolution 220 UV-visible spectrometer (Thermo Scientific, Madison, WI). For solid-state UV-vis spectroscopy, the samples coated on thin film were analyzed in the wavelength range of 300-700 nm.

Fluorescence spectroscopy: The measurements were carried out using a Fluorolog spectrophotometer (HORIBA Scientific, Irvine, CA). For the analysis, thin-film samples were prepared by drop-casting 50 μL of 15 mg/mL solution onto glass substrates; the paper probes were prepared by drop-casting 5 μL of 15 mg/mL solution onto Whatman (grade 1) filter paper.

Fourier-transform infrared spectroscopy (FTIR): The measurements were performed on a Thermo Scientific Nicolet 6700 spectrometer in ATR mode. The sample was prepared by drop-casting 15 mg/mL solution of the desired compound onto glass substrates.

Raman scattering: The measurements were performed on a LabRam HR-high resolution analytical Raman (Horiba Jobin Yvon, France). The excitation source was a 753 nm laser, and 50×long-focal-length objective lenses were employed. The sample was prepared by drop-casting 15 mg/mL of the desired compound solution onto glass substrates.

Scanning electron microscopy (SEM): The images were obtained on a JEOL scanning electron microscope (Tokyo, Japan, JSM-7400F). For SEM imaging, the corresponding samples were coated with gold and imaged at different magnifications.

Nuclear magnetic resonance (NMR): The spectra were recorded on a Bruker DPX 400 spectrometer using CDCl3 as a solvent and tetramethylsilane (TMS) as an internal standard. Chemical shifts are relative to TMS. MestreNova software was used for analyzing the NMR data.

### Example 1

### Synthesis of chalcone-substituted diacetylene monomer bearing protonatable amine group (CHA-DA)

### Part A: Preparation of CHA

A round-bottomed flask was charged with ethanol (10 ml) and 10% sodium hydroxide (5 ml) followed by 4-(dimethylamino)benzaldehyde (745 mg, 5 mmol), and the mixture was stirred to complete dissolution. 1-(4-Hydroxyphenyl) ethan-1-one (816 mg, 6 mmol) was added, and the reaction mixture was stirred at RT for 24 hours. After the reaction, HCl solution was added to quench the reaction, and the aqueous layer was extracted with EA (50 mL×3). The combined organic layers were washed with brine (50 mL), dried over Na₂SO₄ and concentrated. The solvent was removed under reduced pressure and the residue was chromatographed on silica gel (Petroleum Ether/Ethyl acetate) to afford 910 mg of desired product CHA (68% yield).

### Part B: Preparation of CHA-DA

A round-bottomed flask was charged with 10,12-tricosadiynoic acid (TRCDA, 2.6 mmol, 900 mg) and dichloromethane (DCM, 20 mL) to obtain a solution. Oxalyl chloride (0.45 mL, 5.2 mmol) was added, and the mixture was stirred for 30 min at room temperature. N,N-dimethylformamide (DMF, catalytic amounts-2-3 drops) was added to the solution and the reaction mixture was stirred for 4.5 hours. The solvents and excess oxalyl chloride were removed by a rotary evaporator under a vacuum to give acyl chloride of 10,12-tricosadiynoic acid (TRCDA-Cl). The TRCDA-Cl residue was dissolved in DCM (10 mL) and the solution was directly used in the next step.

(E)-3-(4-(dimethylamino)phenyl)-1-(4-hydroxyphenyl) prop-2-en-1-one (CHA, 534 mg, 2 mmol) was dissolved in DCM (10 mL) in a separate flask, followed by the addition of triethyl amine (3 mmol, 0.417 mL). Then the acyl chloride solution (from the previous step) was added dropwise to the reaction mixture and once the addition is completed the resultant solution was stirred at room temperature for another 16 hours. Upon completion of the reaction, the solvent was removed under reduced pressure and the residue was chromatographed on silica gel (Petroleum Ether/Ethyl acetate 75/25) to afford 964 mg of the desired product CHA-DA (81% yield). The compound CHA-DA was obtained as a pale yellow solid and the structure of the compound was confirmed by ¹H, ¹³C NMR and HRMS data. ¹H NMR (400 MHZ, CDCl₃): δ 8.17 (d, J=8.7 Hz, 2H), 7.93 (d, J=15.5 Hz, 1H), 7.69 (d, J=8.8 Hz, 2H), 7.45 (d, J=15.5 Hz, 1H), 7.34 (d, J=8.7 Hz, 2H), 6.85 (d, J=8.6 Hz, 2H), 3.19 (s, 6H), 2.72 (t, J=7.5 Hz, 2H), 2.38 (d, J=6.8 Hz, 4H), 1.94-1.87 (m, 2H), 1.65 (dd, J=14.7, 7.2 Hz, 6H), 1.53 (dd, J=14.0, 7.0 Hz, 8H), 1.39 (s, 13H), 1.02 (d, J=6.6 Hz, 3H). ¹³C NMR (100 MHz, CDCl₃): δ 189.3, 171.8, 145.5, 141.9, 134.6, 134.5, 130.5, 129.9, 119.0, 117.2, 112.6, 112.5, 65.4, 407, 38.0, 32.0, 29.7, 29.6, 29.4, 29.3, 29.2, 29.0, 28.9, 28.5, 28.4, 25.6, 22.8, 19.3, 14.2.

HRMS (ESI): calculated for [(C₄₀H₅₃NO₃)H] (M+H) 596.4104, measured 596.4088.

### Examples 2A-2D (Comparative) and 2E (of the Invention)

### Polymerizability of CHA-DA and acid-treated CHA-DA monomers

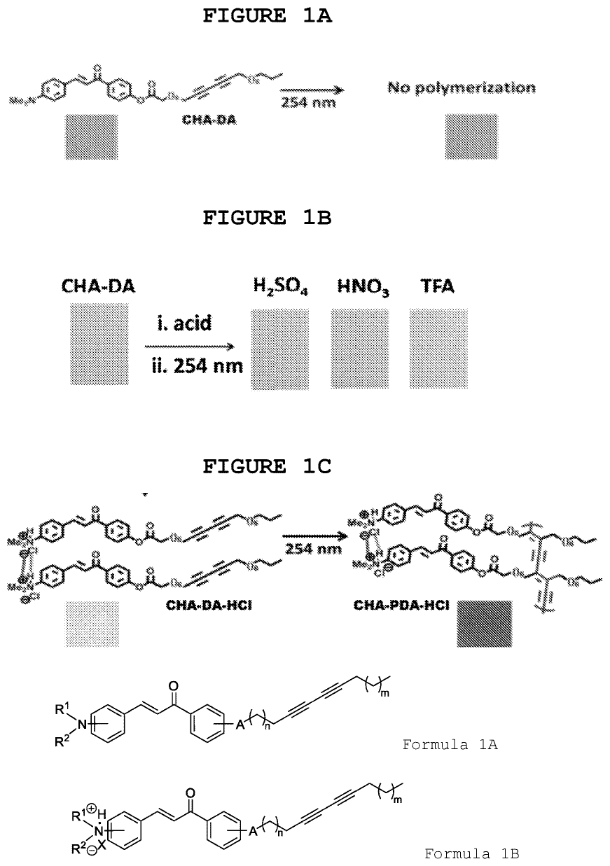

CHA-DA monomer of Example 1 was dissolved in chloroform to form a 15 mg/mL solution. 5 μL of the CHA-DA solution was drop-casted onto Whatman (grade 1) filter paper (1 cm²). Then the resultant, yellow-colored film was dried for two minutes and subjected to UV irradiation at 254 nm for one minute (Example 2A), or exposed to saturated acid vapors for one minute, and then UV irradiated at 254 nm for one min (Examples 2B-2E). The action of four acids on the yellow-colored film CHA-DA film was studied (sulfuric acid, nitric acid, trifluoroacetic acid and hydrochloric acid—Examples 2B-E, respectively) to evaluate the polymerizability of the acid-treated CHA-DA monomer. The results are tabulated in Table 1 and are shown in FIGS. 1A-1C.

TABLE 1

| | Acidic treatment on the yellow- | UV irradiation |
|---|---|---|
| Example | colored CHA—DA casted film | 254 nm |
| 2A | None | No polymerization |
| (comparative) | | occurred |
| 2B | H₂SO₄ vapors; | No polymerization |
| (comparative) | film retained its yellow color | occurred |
| 2C | HNO₃ vapors; | No polymerization |
| (comparative) | film retained its yellow color | occurred |
| 2D | CF₃COOH vapors; | No polymerization |
| (comparative) | A colorless film was obtained | occurred |
| 2E | HCl vapors; | Yes, purple-colored |
| (invention) | a colorless CHA—DA—HCl | CHA—PDA—HCl |
| | film was obtained | film |

The results tabulated in Table 1 indicate that neither the chalcone-diacetylene monomer, nor acid-treated chalcone-diacetylene monomers, when the acid was H₂SO₄, HNO₃, and trifluoroacetic acid, underwent UV polymerization to form colored PDA films, apparently due to the bulkiness of the negative counter ions in those acids. In contrast, exposure of the yellow-colored chalcone-diacetylene monomer to HCl vapors resulted in the formation of a colorless film, consisting of the monomer molecules in the protonated form (the ammonium salt), which in turn underwent polymerization, successfully creating the purple colored PDA network, underscoring the key role of the chloride ions in mediating the reorganization of the acid-treated chalcone-diacetylene monomers.

### Example 3

### Characterization of the CHA-DA-HCl monomer and the corresponding PDA film

The HCl-reacted monomer (i.e., the colorless CHA-DA-HCl film) and its UV polymerization product (CHA-PDA-HCl film) from Example 2E, were studied by scanning electron microscopy (SEM) and spectroscopic methods.

SEM images (not shown) of the monomer CHA-DA, the reaction product of the monomer and gaseous HCl, and the UV polymerization product show distinct structural rearrangements of the diacetylenes, following exposure to HCl and subsequent polymerization.

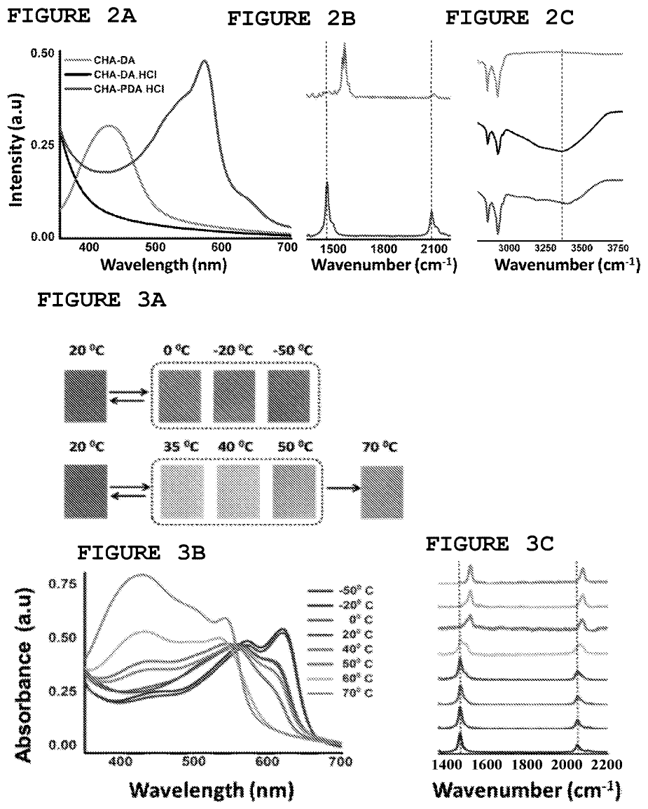

FIGS. 2A-2C depict the spectroscopic characterization of the HCl-treated chalcone-PDA film, corroborating the structural modulation within the chalcone-diacetylene films accounting for the formation of the polydiacetylene network. The UV-vis spectrum of the initial, yellow-colored chalcone-diacetylene monomer in FIG. 2A features a maximum at around 420 nm accounting for absorption in the violet region and the appearance of the corresponding complementary color yellow of the chalcogen moieties. Following exposure to HCl vapor, this peak completely disappeared, attesting to the blocking of charge transfer within the chalcone residue due to the protonation of the dimethylamine unit. The subsequent UV irradiation (at 254 nm) gave rise to the characteristic peak at around 570 nm of purple-colored PDA.

The Raman scattering data in FIG. 2B furnished additional evidence for the structural transformations within the chalcone-diacetylene film. Specifically, the Raman spectrum of the chalcone-diacetylene monomer features a prominent band for acetylene (C≡C) at 2090 cm⁻¹, and a small alkene (C═C) peak at 1560 cm⁻¹. After protonation by the action of the gaseous HCl and subsequent UV irradiation, the alkyne band appeared at 2073 cm⁻¹ and alkene (C═C) peak at 1454 cm⁻¹ reflecting the formation of the conjugated ene-yne polymer network in the chalcone-PDA film.

Fourier transform infrared (FTIR) analysis in FIG. 2C complements the UV-vis and Raman spectroscopy experiments, particularly furnishing insight into the supramolecular organization of the chalcone-diacetylene units. Specifically, in the initial yellow-colored chalcone-diacetylene monomer film, the C—H stretching region in the FTIR spectrum displays two peaks at 2710 cm⁻¹ and 2805 cm⁻¹, ascribed to aliphatic and aromatic C—H stretching peaks, respectively (FIG. 2C, top spectrum). After exposure to HCl vapor, a prominent broad peak appeared at around 3350 cm⁻¹, accounting for the emergence of the protonated N—H band (FIG. 2C, middle spectrum). After polymerization, the N—H band was red-shifted and slightly reduced in intensity, likely corresponding to the hydrogen bond network contributing to photopolymerization and formation of PDA (FIG. 2C, bottom spectrum).

### Example 4

### Thermochromism of the CHA-PDA-HCl film

FIGS. 3A-3C show the thermochromic properties of the HCl-treated chalcone-PDA film that was deposited on filter paper (i.e., of Example 2E).

At room temperature, the film exhibits a purple color. Colorimetric transformations were recorded upon lowering the temperature below 20° C. (FIG. 3A, top row). A purple-blue transition was apparent even upon cooling the HCl-treated chalcone-PDA film down to −50° C. Notably, all color transitions were reversible, with the HCl-treated chalcone-PDA film showing stability after multiple (e.g. twenty) cooling-warming cycles (20 to −50° C.).

Different color changes occurred upon exposing the HCl-treated chalcone-PDA film to high temperatures (FIG. 3A, bottom row). Specifically, upon heating to 50° C., the film underwent a reversible purple-orange transition. But further heating to 70° C., produced an irreversible transformation to a yellow-orange color.

The thermochromic transformations of chalcone-PDA are also manifested in the UV-vis spectroscopy (FIG. 3B) and Raman scattering (FIG. 3C) analyses. Specifically, the prominent visible absorbance peak at around 630 nm with the 580 nm shoulder corresponding to polymerized blue PDA is observed at −50° C. (FIG. 3B). This peak was gradually blue-shifted upon increasing the temperature, reflecting color transformations to purple (at 20° C.) and orange-red at higher temperatures. Importantly, the pronounced chalcone absorbance at 420 nm appears upon heating the film to 70° C., accounting for the occurrence of charge transfer within the chalcone residue upon release of HCl.

Raman spectroscopy data in FIG. 3C further underscore the molecular transformations associated with the thermochromic properties of HCl-treated chalcone-PDA. Specifically, the band at 2087 cm⁻¹ (corresponding to C═C stretching) gradually shifted to a higher frequency at 2115 cm⁻¹, accounting for the thermochromic (blue-to-red) transformations of the film. Raman spectral changes are also apparent in the case of the alkene band at around 1450 cm⁻¹. Specifically, a shoulder at around 1490 cm⁻¹ emerged upon heating from −50° C. to 0° C. (FIG. 3C) reflecting the small conformational change associated with the blue-purple color transition of the PDA system. Further heating of the HCl-treated chalcone-PDA film resulted in the disappearance of the C═C peak at 1450 cm⁻¹, and the emergence of the signal at 1500 cm⁻¹ as the predominant spectral feature accounting for the pronounced blue-red PDA transformation.

### Example 5

### The sensing of NH₃ vapor by the CHA-PDA-HCl film

The goal of the study was to evaluate the detectability of various analytes in the gaseous state by the CHA-PDA-HCl film, i.e., sensing vapors of ammonia vapors and amine compounds.

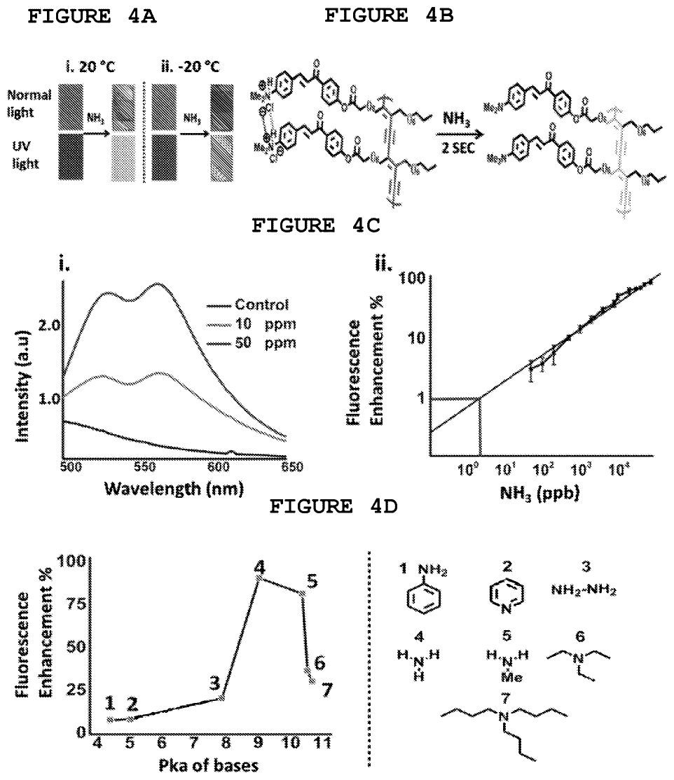

FIG. 4A features the pronounced color (top row) and fluorescence (bottom row) transformations of the HCl-treated chalcone-PDA film upon brief exposure to ammonia gas (200 ppm) at 20° C. and at −20° C. At 20° C., ammonia induced a remarkable purple-orange visible transition (FIG. 4A,i top row) and a blue-yellow fluorescence transformation (excitation at 365 nm; FIG. 4A,i bottom row), while at −20° C., the corresponding ammonia-induced visible color and fluorescence were more greenish/brown (FIG. 4A,ii).

The remarkable optical changes depicted in FIG. 4A are attributed to the removal of the HCl molecules associated with chalcone-PDA, and ammonia-induced structural and associated chromatic transition of the conjugated network of PDA. Both effects are portrayed in the reaction scheme in FIG. 4B. Specifically, scavenging of the acidic proton bound to the dimethylamine groups by ammonia and simultaneous removal of the cognate Cl-ions reintroduce charge transfer from the NMe₂ groups (electron donors) to the carbonyl units (electron acceptors). In parallel, ammonia induced the phase transformation within the conjugated PDA network giving rise to a blue-red transition of PDA. Consequently, a blending of the yellow color of the chalcone residue (due to charge transfer) and red PDA (at 20° C.) gave rise to the dark orange color shown in FIG. 4A,i. Similarly interesting visual transformations were observed in the fluorescence experiments (FIG. 4A,i, bottom). The initial blue appearance reflects the fact that blue-phase PDA is non-fluorescent while the fluorescence of the HCl-treated chalcone is blue. However, following ammonia exposure, the combination of pale-yellow fluorescence of chalcone and red fluorescence of PDA produce the pastel orange color shown in FIG. 4A,i, bottom.

Exposure of the PDA film to ammonia vapors at −20° C. did not give rise to the blue-red transformation of PDA due to the significantly constrained motion of the conjugated network at that temperature. Accordingly, the visible color at that temperature was dark green, arising from the blending of the yellow color of the chalcone unit and the lavender blue PDA (FIG. 4A,ii, top). Similarly, the greenish-yellow fluorescence induced by ammonia in the HCl-treated chalcone-PDA film is ascribed to the mixing of yellow fluorescence of the chalcone units and the blue-purple emitted fluorescence of the purple phase PDA (FIG. 4A,ii, bottom).

In comparison with the HCl-treated chalcone-PDA film, the simple PDA film derived from 10,12-tricosadiynoic acid was less reactive towards ammonia vapors and produced only a small color change (blue to purple-blue transition) with a higher concentration of ammonia (1000 ppm) and longer exposure times (five minutes) at ambient temperature.

FIG. 4C depicts the fluorescence modulation induced upon exposure of the HCl-treated polymerized chalcone-PDA film to ammonia vapor. Fluorescence enhancement after exposure to an analyte was calculated by using the following formula obtained [R. Borah and A. Kumar, Mater. Sci. Eng. C., 2016, 61, 762-772]:

$${F}_{e} = \frac{{F}_{a} - {F}_{b}}{{F}_{a}} \times 100 \qquad \begin{matrix}{F}_{e} = Fluorescence enhancement . \\ {F}_{a} = Fluorescence intensity after exposure . \\ {F}_{b} = Fluorescence intensity before exposure .\end{matrix}$$

The fluorescence emission spectra (excitation 450 nm) in FIG. 4C,i show a significant enhancement of the fluorescence emission, directly dependent upon the concentration of ammonia vapor molecules. Importantly, the calibration curve in FIG. 4C,ii reveals a linear relationship in the range of 0.1-100 ppm, and a detection limit of around 3 ppb (corresponding to experimentally significant 1% fluorescence enhancement), which is on par or better than previously reported ammonia vapor sensors.

FIG. 4D underlines the selectivity of the HCl-treated chalcone-PDA optical sensor film among different amine vapors. In the experiments, HCl-treated chalcone-PDA films were exposed to the indicated amines 1-7 (at 100 ppm concentration), and the fluorescence emission at 568 nm (excitation 450 nm) was recorded. The fluorescence emission intensity trend in FIG. 4D reflects two parameters contributing in tandem to the optical transformation of the HCl-treated chalcone-PDA film. Specifically, lower PKa values in the case of aniline (1), pyridine (2) and hydrazine (3) likely account for low reactivity (i.e., lesser proton scavenging of the HCl-treated dimethylamine) and concomitant lower fluorescence enhancement (FIG. 4D). In the case of triethylamine (6) and tributylamine (7), the bulky residues likely inhibit proton scavenging capabilities by these two amines, similarly minimizing the fluorescence enhancement effect.

In comparison, the relatively high pKa of ammonia and methylamine combined with the steric accessibility of the nitrogen electron lone pair in these two molecules afford reactivity and concomitant pronounced fluorescence enhancement induced in the HCl-treated chalcone-PDA. It should also be emphasized, that no color/fluorescence changes for the HCl-treated polymerized chalcone-PDA film were observed after exposure to various organic vapors, including hexane, toluene, DCM, chloroform, THF, DMF, DMSO and EtOH.

### Example 6

### The Sensing of NH₃ Generated by Bacteria with the CHA-PDA-HCl Film

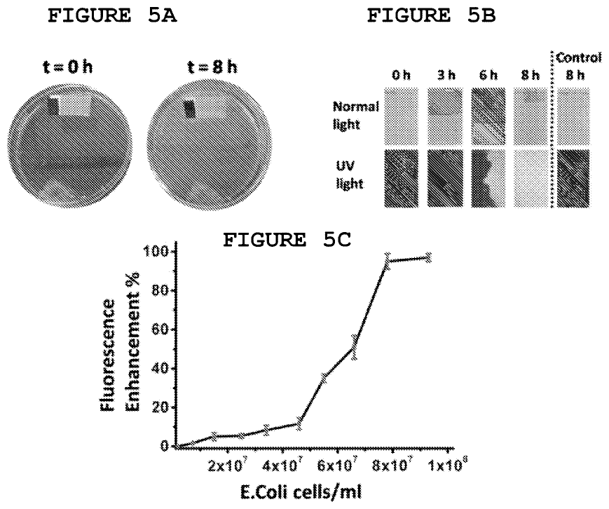

Ammonia is a prominent volatile metabolite secreted by bacteria. Accordingly, the HCl-treated chalcone-PDA films were tested for visual bacterial sensing (FIGS. 5A-5C). To this end, bacterial strains were cultured in Luria-Bertani (LB) medium at 37° C. Single bacterial colonies from LB agar plates were added to 10 mL of LB broth and maintained at 37° C. for 12h in a shaking incubator (220 rpm). The concentration of bacteria in the medium was determined by measuring the optical density at 600 nm (OD 600). For the sensing experiment, 1.6*106 E. coli bacterial cells in a Luria-Bertani (LB) medium were grown on a Petri dish at a constant temperature (37° C.). The gas emissions from bacteria were monitored by placing a chalcone-PDA deposited paper probe 1 cm above the bacterial solution in a Petri dish (on the cover of the Petri dish). The corresponding color changes from the paper probe were recorded at different time intervals.

At 37° C., the HCl-treated chalcone-PDA film was initially light-purple but transformed to an orange color within eight hours, accounting for the ammonia gas released by the proliferating bacteria in the LB medium (see FIG. 5A).

The photographs in FIG. 5B further show the gradual color and fluorescence changes of the HCl-treated chalcone-PDA film. Notably, distinguishable color/fluorescence transformations could be discerned within 3 to 6 hours after initiation of bacterial growth. FIG. 5B also confirms that the control chalcone-PDA film did not undergo chromatic transformations in the presence of LB solution that was not spiked with bacteria. The fluorescence response curve in FIG. 5C (calculated as previously described) further attests to the correlation between the chromatic changes of the film and bacterial proliferation, underscoring the typical exponential growth curve of bacterial populations.

### Example 7

### The Sensing of Food Spoilage by the CHA-PDA-HCl Film

The goal of the study was to evaluate the ability of the CHA-PDA-HCl film to monitor food spoilage processes, through the detection of volatile ammonia generated by bacteria proliferating in food products.

In the food spoilage tests, 10 gm of store-purchased fresh-cut fish (Sparus aurata), chicken and beef were placed in the Petri dish and a paper-deposited chalcone-PDA film was attached to the top cover of the plate. Color and fluorescence monitoring was carried out both at 25° C. (room temperature conditions) and 4° C. (refrigerated conditions).

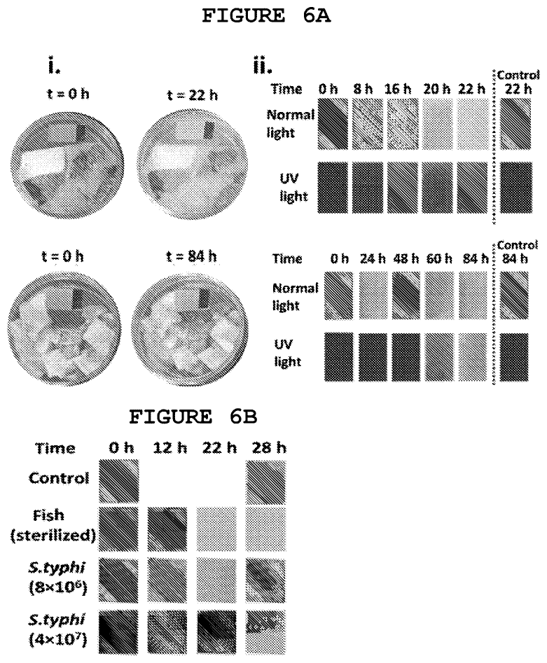

The results shown in FIG. 6 indicate that the HCl-treated chalcone-PDA film underwent a pronounced visible purple-orange transformation within 22 hours (fish at room temperature, FIG. 6A,i) or 84 hours (fish at 4° C., FIG. 6A,ii), accounting for bacterial proliferation in the fish sample.

The time-dependent color and fluorescence photographs of the fish-exposed film in FIG. 6A,ii attest to the feasibility of employing the platform for the HCl-treated chalcone-PDA system for visual monitoring of food spoilage. At room temperature, visual color and fluorescence changes could be discerned already within 16 hours, while after 20 hours a pronounced optical transformation of the film was readily observed, indicating significant bacterial proliferation in the fish sample (FIG. 6A,ii). Importantly, the HCl-treated chalcone-PDA films could be similarly employed for visible monitoring of food spoilage at 4° C. (FIG. 6A,ii, bottom row). Specifically, at 4 0C the color/fluorescence transformations occurred within ˜48 hours, reflecting the slower proliferation of bacteria at this temperature. Similar color/fluorescence transformations as presented in FIG. 6A were recorded in experiments utilizing beef and chicken.

To further confirm that the striking visual transformations of the HCl-treated chalcone-PDA films in the food samples (e.g., FIG. 6A) are indeed induced by bacterial contamination, a bacterial spiking experiment was carried out, in which different bacterial concentrations were added to sterilized fish samples (i.e., boiled for 15 minutes) and monitored the color changes of HCl-treated chalcone-PDA films placed above the samples (FIG. 6B). The color photographs in FIG. 6B indeed demonstrate that the purple-orange color change of the HCl-treated chalcone-PDA film was observed earlier in the case of a fish sample spiked with a higher bacterial quantity. Importantly, no color transformation occurred, for the duration of measurement, in the sterilized fish sample to which no bacteria were added.

While the present disclosure has been illustrated and described with respect to a particular embodiment thereof, it should be appreciated by those of ordinary skill in the art that various modifications to this disclosure may be made without departing from the spirit and scope of the present disclosure.

What is claimed is:

1. An aminochalcone-diacetylene compound of Formula 1A: 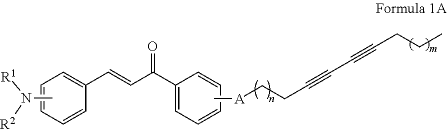
wherein R¹ and R² are independently selected from H and C₁-C₃ alkyl, A denotes a linkage connecting the chalcone and diacetylene units, m and n are independently integers in the range of 2 to 18, and the corresponding protonated form/ammonium salt of Formula 1B: 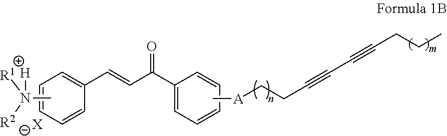
wherein X is a counter anion.

2. A compound of Formula 1A or 1B according to claim 1, wherein the linkage A comprises an ester bond or an amide bond.

3. A compound of Formula 1A or 1B according to claim 2, wherein the linkage A is an ester bond.

4. A compound of Formula 1A or 1B according to claim 1, wherein the linkage A and the —NR¹R² group are both at the para positions of the respective rings.

5. The compound of Formula 1B according to claim 1, wherein X is chloride.

6. A compound of Formula 1A or 1B according to claim 1, wherein the compound is N,N-dialkylated.

7. The compound of Formula 1A according to claim 1, which is

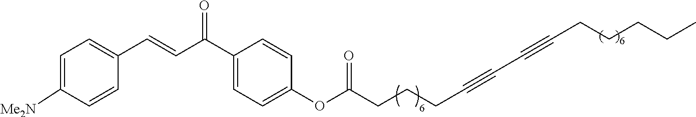

8. A process for preparing a compound of Formula 1A comprising reacting a diacetylene compound of Formula 2 and aminochalcone of Formula 3: 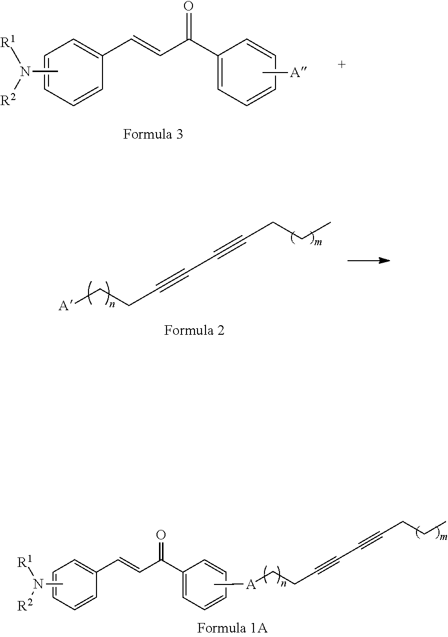
wherein n, m, R¹, R² and A are as defined in claim 1, A′ and A″ are functional groups which participate in a linkage formation reaction, to create a linkage A.

9. The process according to claim 8, comprising reacting an alcohol of Formula 3, wherein A″ is —OH, and an acyl chloride of Formula 2, wherein A′ is —C(O)Cl, in an organic solvent in the presence of amine catalyst, to form the corresponding ester of Formula 1A, wherein A is —O—C(O).

10. A process comprising:
assembling a chalcone-diacetylene of Formula 1A into a thin film;
treating the thin film with gaseous acid, to form the compound of Formula 1B; and
photopolymerizing the compound of Formula 1B to afford the corresponding polydiacetylene of Formula 5: 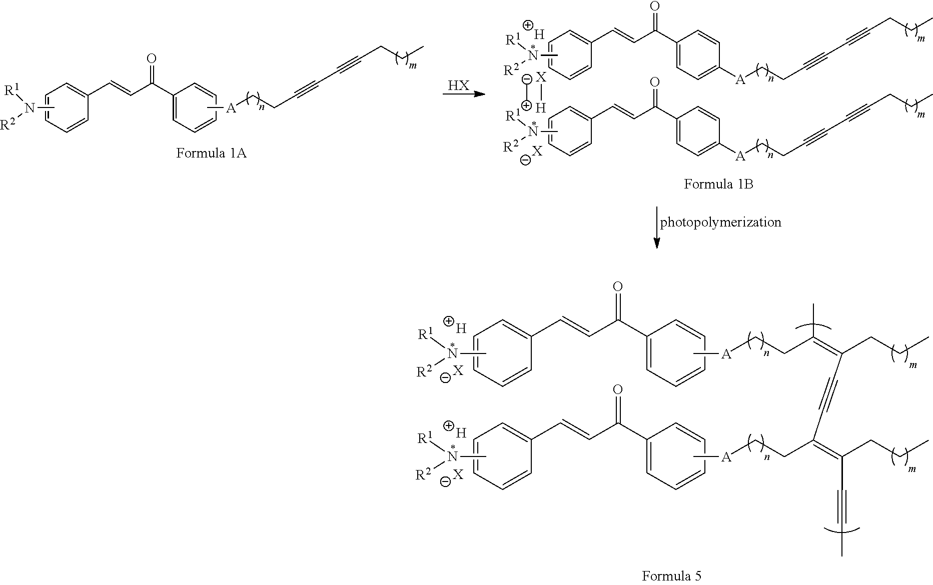
wherein R¹ and R² are independently selected from H and C₁-C₃ alkyl, A is an ester bond or an amide bond, m and n are integers in the range of 2 to 18 and X is a counter anion.

11. A chalcone-polydiacetylene of Formula 5: 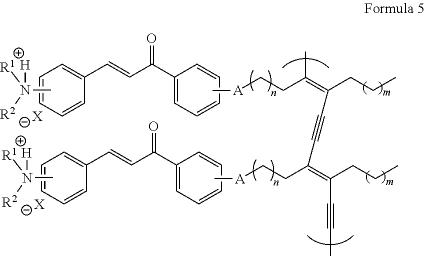
wherein R¹ and R² are independently selected from H and C₁-C₃ alkyl, A is an ester bond or an amide bond, m and n are integers in the range of 2 to 18 and X is a counter anion.

12. The chalcone-polydiacetylene of Formula 5 according to claim 11, wherein R¹ and R² are both methyl, A is an ester bond —O—C(O)—, wherein the linkage A and the —NR¹R² group are both at the para positions of the respective rings, n equals 6, m equals 8 and X is chloride.

13. A colorimetric and/or fluorescent sensor for detection of vapors of ammonia and related amine compounds, comprising the chalcone-polydiacetylene of Formula 5 as defined in claim 11.

14. A colorimetric and/or fluorescent sensor for detection of biogenic ammonia and related amines compounds, comprising the chalcone-polydiacetylene of Formula 5 as defined in claim 11.

15. A colorimetric and/or fluorescent sensor for monitoring food spoilage, comprising the chalcone-polydiacetylene of Formula 5 as defined in claim 11.
